(19) United States
(12) Patent Application Publication
(10) Pub. No.: US 2007/0142880 A1
(43) Pub. Date: Jun. 21, 2007

(54) Light delivery apparatus

(76) Inventors: William Louis Barnard, Maple Valley, WA (US); Phillip Burwell, Snohomish, WA (US); James C. Chen, Bellevue, WA (US); Julene S. Christophersen, Carnation, WA (US); William L. Gembala, Newcastle, WA (US); David B. Shine, Sammamish, WA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 11/594,570

(22) Filed: Nov. 7, 2006

Related U.S. Application Data
(60) Provisional application No. 60/734,184, filed on Nov. 7, 2005.
(60) Provisional application No. 60/762,407, filed on Jan. 26, 2006.
(60) Provisional application No. 60/851,102, filed on Oct. 11, 2006.

(51) Int. Cl. A61N 5/06

(52) U.S. Cl. 607/88

(57) ABSTRACT

A light delivery apparatus to provide light treatment to a patient includes a catheter assembly having a light source that transmit light towards a target site within a patient. A balloon surrounds the light source and has a plurality of tissue engaging elements movable between first positions and second positions. In one embodiment, each element extends radially inward in the first position and radially outward in the second position. An insertion tool is used to deliver the light delivery apparatus. During use, the light delivery apparatus is rotationally locked with the insertion tool for improved steering, navigation, and aiming of emitted light.

### CROSS-REFERENCES TO RELATED APPLICATIONS

This application claims the benefit under 35 U.S.C. § 119(e) of U.S. Provisional Patent Application No. 60/734,184 filed Nov. 7, 2005; U.S. Provisional Patent Application No. 60/762,407 filed Jan. 26, 2006; and U.S. Provisional Patent Application No. 60/851,102 filed Oct. 11, 2006; where these three provisional applications are incorporated herein by reference in their entireties.

### BACKGROUND OF THE INVENTION

1. Field of the Invention

The present invention relates generally to a light delivery system useable for medical treatment, such as light therapy.

2. Description of the Related Art

Typical photodynamic therapy (“PDT”) employs light to treat or investigate photosensitized tissues. A photoreactive or photosensitizing agent having a characteristic light absorption waveband is typically administered to the patient, either orally or by injection or even by local delivery to the treatment site. The photoreactive or photosensitizing agent is subsequently selectively absorbed by abnormal tissue much more so than by normal tissue. Once the abnormal tissue has absorbed or linked with the photoreactive or photosensitizing agent, the abnormal tissue can then be destroyed by administering light of an appropriate wavelength or waveband corresponding to the absorption wavelength or waveband of the photoreactive agent. PDT has proven effective in destroying abnormal tissue, such as cancer cells. Traditional PDT is often unsuitable for performing highly localized treatments on, for example, vascular targets largely because typical methods of delivering the photoreactive agent do not permit accurate localization of the PDT.

The objective of PDT may be either diagnostic or therapeutic. In diagnostic applications, the wavelength of light is selected to cause the photo-reactive agent to fluoresce as a means to acquire information about the targeted cells without damaging the targeted cells. In therapeutic applications, the wavelength of light delivered to the targeted cells treated with the photo-reactive agent causes the agent to undergo a photochemical reaction with oxygen in the localized targeted cells, to yield free radical species (such as singlet oxygen), which cause localized cell lysis or necrosis.

PDT has therefore proven to be an effective oncology treatment for destroying targeted cancerous cells. In addition, PDT has been proposed as a treatment for other ailments, some of which are described in Applicant's co-pending patent application U.S. Publication No. 2005/0228260 (U.S. patent application Ser. No. 10/799,357, which is hereinafter referred to as the '357 patent application).

One type of light delivery system used for PDT treatments comprises the delivery of light from a light source, such as a laser, to the targeted cells using a single optical fiber delivery system with special light-diffusing tips. This type of light delivery system may further include single optical fiber cylindrical diffusers, spherical diffusers, micro-lensing systems, an over-the-wire cylindrical diffusing multi-optical fiber catheter, and a light-diffusing optical fiber guidewire. This light delivery system generally employs a remotely disposed high-powered laser or solid state laser diode array, coupled to optical fibers for delivery of the light to the targeted cells. However, the use of laser light sources has several drawbacks, such as relatively high capital costs, relatively large size equipment, complex operating procedures, and safety issues in working with and around high-powered lasers.

The '357 patent application addresses some of these concerns and also addresses the desire to develop a light-generating apparatus that can be secured within a blood vessel or other orifice. The securing mechanism of such an apparatus would also be capable of removing light absorbent or light blocking materials, such as blood, tissue, or another object from the light path between the targeted cells and the light transmitters. Securing the apparatus within a blood vessel, for example, can be achieved with an inflatable balloon catheter that matches the diameter of the blood vessel when the balloon is inflated.

An introducing sheath having a lumen extending therethrough to create a passageway for insertion of other instruments into a patient's body through the sheath may be used with the light delivery system. One type of introducing sheath is described in another one of Applicant's co-pending patent applications, PCT Application No. PCT/US2005/032851. In general, this type of introducing sheath surrounds a penetrating device, which is introduced into the body and then removed, leaving the sheath behind as a passageway. One such instrument that can be inserted through the sheath is a light catheter for PDT treatment.

The light source for the light system used for PDT treatments may also be light emitting diodes (LEDs). Arranged LEDs form a light bar for the light system, where the LEDs may be either wire bonded or electrically coupled utilizing a “flip-chip” technique that is used in arranging other types of semiconductor chips on a conductive substrate. Various arrangements and configurations of LEDs are described in U.S. Pat. Nos. 6,958,498; 6,784,460; and 6,445,011; and also in the '357 patent application.

### BRIEF SUMMARY OF THE INVENTION

The embodiments described herein are generally related to a treatment system usable for treating one or more internal target sites. By using minimally invasive techniques, the light delivery system can treat target sites at different depths and positions in an individual's body. The target sites can include, without limitation, diseased tissues (e.g., cancerous cells), interstitial tissues, epithelial tissues, connective tissues (e.g., blood, cartilage, and/or bone), nerve tissues, or other regions of interest. The target site can be treated with or without using medicaments or treatment agents. For example, the disclosed embodiments can treat at least a portion of the omentum to destroy omentum fat, with or without utilizing photosensitive agents, or other energy activated agents. Other types of fat, such as visceral fat, can also be targeted.

The embodiments described herein are generally related to a light delivery system usable for treating a patient by light therapy. Light can be applied externally and/or internally and used to treat various types of medical conditions, such as proliferative diseases or obesity.

The light delivery system can have a distal tip with an expandable member, such as a balloon. In some embodiments, the balloon has at least 1 deployable tissue engaging element. In some embodiments, the balloon has at least 3 deployable tissue engaging elements. In some embodiments, the balloon has at least 5 deployable tissue engaging elements. In some embodiments, the balloon has at least 10 deployable tissue engaging elements. In some embodiments, the balloon has at least 15 deployable tissue engaging elements.

In some embodiments, a device for performing a medical treatment is provided. The device comprises a catheter body configured for placement in a patient and a distal tip coupled to the catheter body and capable of emitting a sufficient amount of light to perform light therapy, the distal tip having an expandable balloon that transmits light for treating the patient, the expandable balloon having a main body and at least one deployable tissue engaging element connected to the main body, the at least one deployable tissue engaging element extends outwardly from the main body when the balloon is inflated.

In some embodiments, a method of treating a patient is provided. The method comprises placing an expandable distal end of a catheter within the patient. The expandable distal end is inflated while the distal end is within the patient. At least one tissue engaging element is deployed such that the at least one element extends outwardly to engage tissue of the patient. A sufficient amount of light is emitted from the expandable distal end to treat target cells in the patient.

In some embodiments, a device for performing light therapy comprises a catheter body and a distal tip. The catheter body is configured for placement in a subject. The distal tip is coupled to the catheter body. The distal tip includes a light source and an expandable balloon. The light source is capable of emitting a sufficient amount of light through the balloon to perform light therapy. The balloon has a main body and at least one deployable tissue engaging element connected to the main body. The at least one deployable tissue engaging element extends outwardly from the main body when the balloon is expanded.

In some embodiments, the light delivery system comprises an insertion device and a light delivery apparatus. The insertion device has a distal end for placement in a subject and a longitudinally extending working lumen extending proximally from the distal end. The light delivery apparatus is dimensioned so as to fit within the working lumen. The light delivery apparatus is coupled to the working lumen such that the light delivery apparatus is substantially rotationally locked with respect to the insertion device while being slidably coupled with the working lumen in an axial direction. Because the light delivery apparatus is rotationally locked with the insertion tool, there is improved steering, navigation, and/or aiming of emitted light.

In yet other embodiments, the device for performing light therapy comprises a catheter body configured for placement in a patient, an activatable light source configured to output a therapeutic amount of light energy, and an expandable member coupled to the catheter body and surrounding the activatable light source. The expandable member is movable between a first configuration and a second configuration. The device also comprises a reflector coupled to the expandable member. The reflector faces the activatable light source such that, when the expandable body is in the second configuration, light energy delivered from the light source is reflected by the reflector.

In some embodiments, a method of treating a subject is provided. The method comprises placing an expandable member in the subject. The expandable member is coupled to a catheter body. A reflector coupled to the expandable member is positioned with respect to an activatable light source disposed within the expandable member. Light from the light source is delivered towards the reflector. Light from the activatable light source is reflected with the reflector such that the reflected light passes through a transparent section of the expandable member.

In some embodiments, a light delivery apparatus for treating a subject is provided. The apparatus comprises a flexible elongate device and an activatable light source. The elongate device is dimensioned for placement in a body of the subject. The elongate device has a first configuration and a selected second configuration, wherein the elongate device in the first configuration is adapted to occupy a first volume. The elongate device in the second configuration is adapted to occupy a second volume that is greater than the first volume. The activatable light source is positioned with respect to the elongate device such that, when the elongate device is in the second configuration, light from the activatable light source can illuminate tissue adjacent at least a portion of the elongate device.

In some embodiments, a method of performing light therapy is provided. The method comprises positioning a flexible elongate device in a subject. The elongate device is moved from a first configuration to a selected second configuration. The elongate device in the first configuration is adapted to occupy a first volume. The elongate device in the second configuration is adapted to occupy a second volume that is greater than the first volume. Light energy is delivered from a light source through at least a portion of the elongate device in the second configuration.

In yet other embodiments, a method of treating target tissue of a subject is provided. The method comprises advancing an illumination device into the subject. The illumination device has at least one light source adapted to emit light capable of activating a treatment agent. The illumination device is positioned in a space in the subject. The space is defined between a first layer of tissue and a second layer of tissue. The illumination device is moved from a first configuration to a selected second configuration. The first layer of tissue is illuminated with light from the at least one light source.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles may not be drawn to scale, and some of these elements may be arbitrarily enlarged and positioned to improve drawing legibility.

FIG. 1 is a side elevational view of a light delivery apparatus having an expandable distal tip, according to one illustrated embodiment.

FIG. 2A is a side elevational view of the distal tip of FIG. 1 where the distal tip is in a collapsed configuration.

FIG. 2B is cutaway view of the distal tip of FIG. 2A where the distal tip has inwardly extending tissue engaging elements disposed around a light source.

FIG. 2C is a side elevational view of the distal tip of FIG. 2A in an expanded configuration.

FIG. 3A is a side elevational view of a portion of the distal tip of FIG. 1 that is positioned near a patient's tissue.

FIG. 3B is a side elevational view of a deployed tissue engaging element extending into a patient's tissue.

FIGS. 3C and 3D are side elevational views of tissue engaging elements having markers to aid in visualization.

FIG. 4A is a side elevational view of a distal tip that includes an array of tissue engaging elements in a first position.

FIG. 4B is a side elevational view of the distal tip of FIG. 4A where the tissue engaging elements are in a second position.

FIG. 5A is a side elevational view of a distal tip having elongate tissue engaging elements.

FIG. 5B is a cross-sectional view of the distal tip of FIG. 5A taken along the line 5B-5B.

FIG. 6A is a side elevational view of a perforated distal tip in accordance with one embodiment.

FIG. 6B is a cross-sectional view of the distal tip of FIG. 6A taken along the line 6B-6B.

FIG. 7A is a side elevational view of a light delivery system having an insertion tool loaded with a light delivery apparatus.

FIG. 7B is a cross-sectional view of the delivery system of FIG. 7A taken along the line 7B-7B.

FIG. 7C is a side elevational view of the delivery system of FIG. 7A where a distal end of the light delivery apparatus protrudes from a window of the insertion tool.

FIGS. 8A to 8C are cross-sectional views of delivery systems having light delivery apparatuses rotationally fixed relative to the insertion tools.

FIG. 9 is a side elevational view of an insertion tool according to one embodiment.

FIG. 10 is a perspective view of a light delivery apparatus having an expandable distal tip, according to one illustrated embodiment.

FIG. 11 is a top view of the light delivery apparatus of FIG. 10.

FIG. 12 is a cross-sectional view of the expandable distal tip of FIG. 10 taken along the line 12-12, according to one illustrated embodiment.

FIG. 13 is a cross-sectional view of the expandable distal tip of FIG. 10 taken along the line 12-12, according to one illustrated embodiment.

FIG. 14 shows a light delivery system performing light therapy on an internal organ.

FIG. 15 is a plan view of a light source having two sets of light emitting devices, wherein one of the sets is activated.

FIG. 16 is a plan view of a light source having two sets of light emitting devices, wherein one of the sets is activated.

FIG. 17 is a plan view of a light source having two sets of light emitting devices, wherein both sets of the light emitting devices are activated.

FIG. 18 is an axial cross-sectional view of a distal tip having a one-sided light bar, according to one illustrated embodiment.

FIG. 19 is an axial cross-sectional view of a distal tip having a two-sided light bar, according to one illustrated embodiment.

FIGS. 20 to 22 are top plan views of an expandable assembly, according to one illustrated embodiment.

FIG. 23 is a side elevational view of a deployable distal tip in a collapsed configuration, according to one illustrated embodiment.

FIG. 24 is a side elevational view of a deployable distal tip in an expanded configuration, according to one illustrated embodiment.

FIG. 25 is a side elevational view of a distal end of the distal tip of FIG. 24.

FIG. 26 shows an insertion device positioned in tissue and a light delivery apparatus in the insertion device, according to one illustrated embodiment.

FIG. 27 shows a partially deployed distal tip of the light delivery apparatus extending from the insertion device of FIG. 26.

FIG. 28 shows the fully deployed distal tip of FIG. 27.

FIG. 29 is a side elevational view of a distal tip of a light delivery apparatus, according to one illustrated embodiment.

FIG. 30 is a side elevational view of a distal tip of a light delivery apparatus, according to one illustrated embodiment.

FIG. 31 is a side elevational view of a straight distal tip of a light delivery apparatus, according to one illustrated embodiment.

FIG. 32 is a cross-sectional view of the distal tip of FIG. 31 taken along the line 32-32.

FIG. 33 is a cross-sectional view of the distal tip of FIG. 31 taken along the line 33-33.

FIGS. 34-37 show distal tips in expanded configurations.

### DETAILED DESCRIPTION OF THE INVENTION

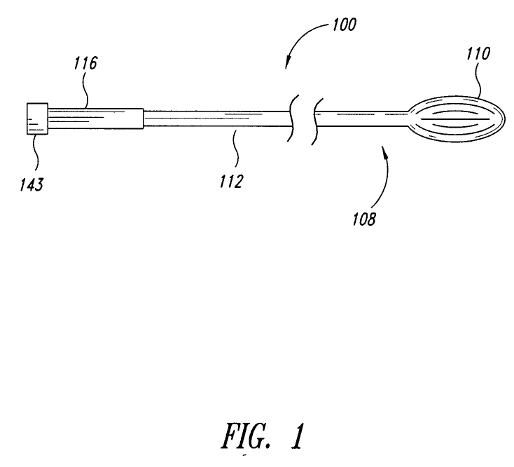

FIG. 1 shows a light delivery apparatus 100 suitable for performing light therapy. As used herein, the term “light therapy” is to be construed broadly to include, without limitation, methods of treating a patient with light with or without treatment agents. Light therapy can be used to treat various types of medical conditions, such as proliferative diseases (e.g., cancer), fat related conditions (e.g., obesity), diabetes, and the like.

The illustrated light delivery apparatus 100 includes an expandable distal tip 110, a connector system 116, and a catheter body 112 extending between the distal tip 110 and connector system 116. The distal tip 110 can be placed in a patient for photo-activating or photo-exciting one or more target cells by subjecting the target cells to a wavelength of light that is emitted from the distal tip 110. The apparatus 100 can be used to perform the treatment methods disclosed in U.S. Pat. Nos. 5,800,478; 6,445,011; and the '357 patent Application, for example.

One or more light sources for emitting light that is suitable for treating the target tissue can be disposed within or near the distal tip 110. Light emitted from the one or more light sources can pass through the distal tip 110 to the target tissue. In this manner, the target tissue can be subjected to at least one wavelength of light that is approximately close to, if not the equivalent to, at least one excitation wavelength of the target tissue, according to some embodiments. It is understood that even if one cell is targeted, it is possible that other cells in the vicinity of the targeted cell may also be subjected to light emitted from the one or more light sources. It is contemplated that the distal tip 110 can be expanded or contracted during, before, and/or after the activation of the light sources.

The elongate catheter body 112 of FIG. 1 can include one or more lumens which provide fluid communication between the connector 116 and distal tip 110. In the illustrated embodiment of FIG. 1, the elongate catheter body 112 comprises a single lumen that extends axially between an interior chamber of the distal tip 110 and the connector 116.

A fluid source can be coupled to the proximal end 143 of the connector 116 to inflate the distal tip 110. Pumps (e.g., syringe pumps), fluid lines, pressurized fluid containers, inflation medium containers, or other types of flowable substance delivery systems can be used to deliver pressurized fluid into the distal tip 110 as discussed in more detail below.

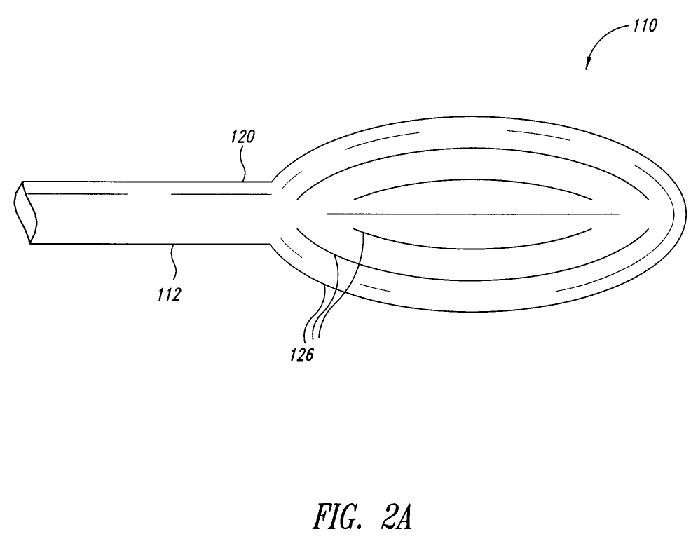

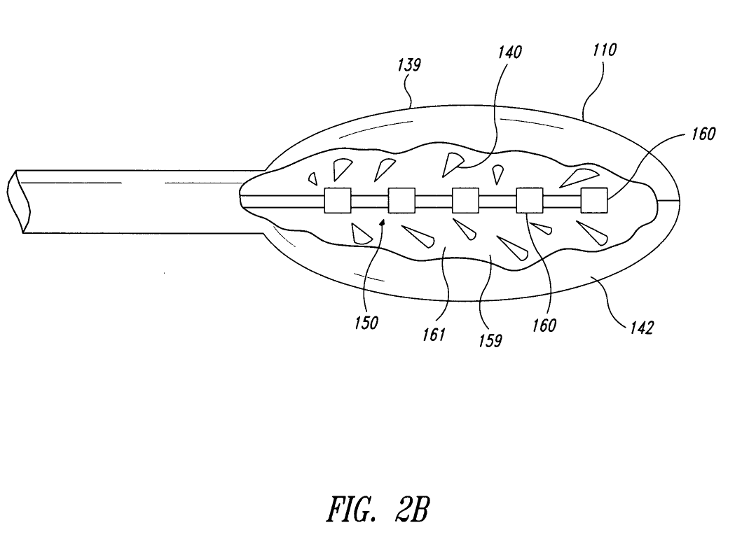

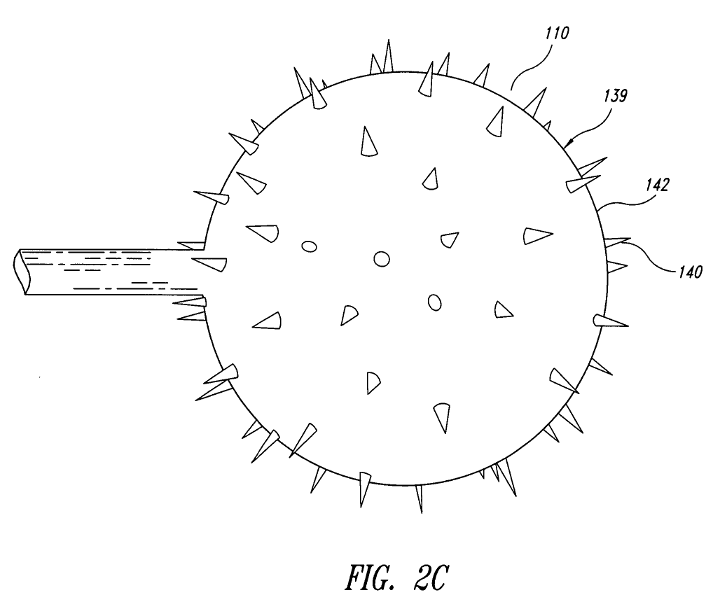

FIG. 2A shows the distal tip 110 mounted to a distal end 120 of the catheter body 112. The distal tip 110 can be inflated from a deflated state (FIG. 2A) to an inflated state (FIG. 2C) by utilizing an inflation fluid.

For insertion and advancement through a patient, the distal tip 110 is preferably in the deflated state for convenient delivery through delivery sheaths, introducers, trocars, insertion devices or tools, intruding sheaths, and the like. However, the distal tip 110 can also be positioned within the patient without using a separate delivery tool. A series of folds 126 can be formed when the distal tip 110 is in its deflated state.

Once the distal tip 110 is positioned within the patient, fluid can be delivered through the connector system 116 and catheter body 112 and into the distal tip 110. The fluid fills and inflates the distal tip 110 to a desired expanded configuration. The distal tip 110 preferably contacts and applies pressure to at least a portion of the target tissue.

FIG. 2B is a cutaway view of a collapsed distal tip 110 having one or more inwardly extending deployable tissue engaging elements 140 formed on a balloon 139. An inner surface 159 of the balloon 139 defines a chamber 161. A light source 150 is disposed within the chamber 161.

The light source 150 can include one or more light emitting elements or energy sources 160. As used herein, the term “energy source” is a broad term and includes, but is not limited to, energy sources capable of emitting radiant energy, such as electromagnetic energy. Non-limiting exemplary energy sources can be light sources capable of emitting visible light waves, non-visible light waves, and combinations thereof. The energy sources can be LEDs (such as edge emitting LEDs, surface emitting LEDs, super luminescent LEDs), laser diodes, or other suitable energy sources. The light emitting elements 160 are preferably LEDs positioned axially along at least a portion of the distal tip 110. U.S. patent application '357 and U.S. Pat. Nos. 5,800,478 and 7,018,395 disclose various types of light emitting devices and elements that can be utilized in the distal tip 110. Each of these references is incorporated by reference in its entirety. In some embodiments, the light source can be in the form of one or more light bars, light strips, and the like.

FIG. 2C shows the distal tip 110 in its distended configuration. The balloon 139 includes a main body 142 and the array of tissue engaging elements 140 extending outwardly from the main body 142. In some embodiments, including the illustrated embodiment, the tissue engaging elements 140 are conical protrusions that extend generally radially outward from a substantially spherically shaped main body 142. However, the tissue engaging elements 140 can extend outwardly at other orientations. Additionally, the elements 140 and main body 142 can have other configurations. For example, the main body 142 can have other curved shapes, including, but not limited to, ellipsoidal, ovoidal, tear, or a combination of arcuate features.

To deploy the distal tip 110, pressurization fluid flows into the chamber 161 of the distal tip 110, as noted above. As the fluid fills the distal tip 110, it causes the tissue engaging elements 140 to be displaced outwardly until they protrude from the main body 142, much like the fingers of a glove being turned inside out. The pressure in the distal tip 110 can be increased or decreased to increase or decrease, respectively, the stiffness of the partially or fully expanded balloon.

Saline, water, gels, diffusers (e.g., diffusing fluids), scattering medium, inflation media, light guide liquids, flowable substances, and other biocompatible materials may be delivered into the distal tip 110. These materials may enhance light distribution by diffusing the emitted light to produce a generally uniform light field. These flowable materials may desirably help distribute heat (e.g., heat generated by the light source) to limit localized overheating. Based on the configuration of the distal tip 110, one of ordinary skill in the art can select an appropriate inflation medium to deploy the distal tip 110 while also providing the desired optical and thermal properties.

The balloon 139 can be made from a polymer, rubber, or other suitable material, preferably a biocompatible material. In some embodiments, the balloon may be formed from one or more of the following materials: polyethylene, polyethylene terephthalate (PET), polypropylene, polyurethane, silicone, a hydrometer urethane, mylar, optical plastics or polymers, light guide materials, or durometer silicone rubber. Of course, other materials can also be used. One of ordinary skill in the art can select the materials to achieve the desired inflation pressure and structural properties needed for adequately penetrating or otherwise engaging the patient's tissue, as discussed below.

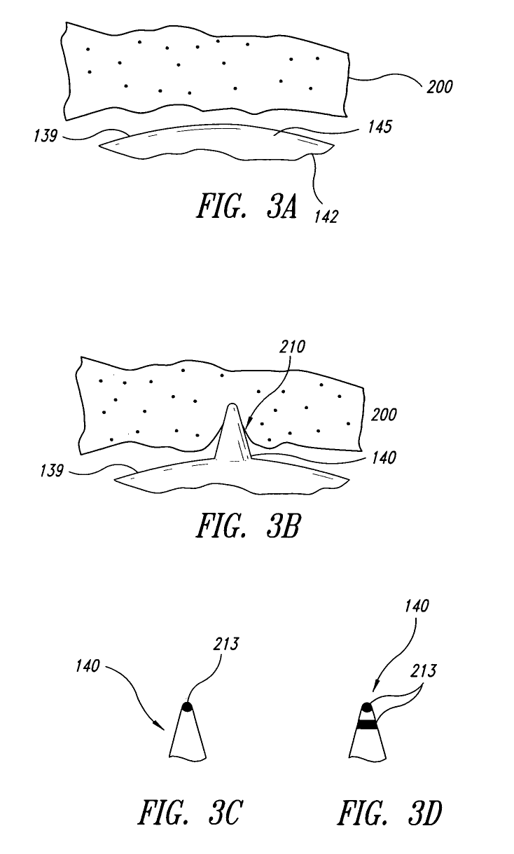

FIG. 3A illustrates a portion of the main body 142 of the balloon 139 that defines a generally smooth, atraumatic surface 145, which can minimize or prevent trauma to tissue, even when the distal tip 110 is partially or fully expanded. FIG. 3B illustrates one of the deployed tissue engaging elements 140 extending outwardly into a target site 210 of the tissue 200. In this manner, the balloon 139 can selectively apply pressure or penetrate tissue (preferably softer tissue such as adipose tissue), while limiting or preventing pressure applied to surrounding organs, membranes, capsules, and the like. The tissue engaging elements 140 can have somewhat blunt or rounded tips for keeping damage to organs or other tissue at or below a desired level. To limit damage to blood vessels, for example, each of the tissue engaging elements 140 can terminate in a blunt tip.

The tissue engaging element 140 of FIG. 3B can advantageously increase the surface area of the illuminated target site 210. The tissue engaging element 140 can push against and stretch the tissue 200, for example. Additionally, if the targeted cells are located deep within tissue, the applied pressure can reduce the distance between the surface of the tissue 200 and the targeted cells. In some embodiments, one or more of the tissue engaging elements 140 can be inserted into natural body lumens or other anatomical structures (e.g., body cavities), between organs, or in other locations suitable for light therapy. Because tissue tends to scatter light, especially in wavebands suitable for PDT, the tissue engaging elements 140 and natural optical properties of the tissue ensure that light is properly distributed, preferably evenly distributed, through the target site.

If desired, the tissue engaging elements 140 can be drawn into the chamber 161 for removing or repositioning of the distal tip 110. A negative pressure can be applied to the connector 116 in order to collapse the distal tip 110 and/or draw in the tissue engaging elements. In this manner, the distal tip 110 can be repeatedly inflated and deflated for any number of treatments.

In operation, visualization techniques can be used to view the position of the distal tip 110. Fluoroscopy (e.g., x-ray fluoroscopy), CT machines, angiography, laparoscopic image guidance systems, or other suitable visualization systems can be used to view the distal tip 110. Visualization media (e.g., radio-opaque fluid, saline, radio-opaque dye, echogenic fluid, combinations thereof, etc.) is preferably used to facilitate proper viewing. In some embodiments, the distal tip 110 is filled with a radio-opaque which is readily visible under fluoroscopy. In this manner, a physician can accurately determine the position of the distal tip 110 within the patient in real time. The tissue engaging elements 140 filled with visualization fluid can also indicate the outer boundaries of the light treatment. Visualization can be used to determine an appropriate deployment position, inflation rate, and pressure to limit or avoid dissections or other types of tissue damage.

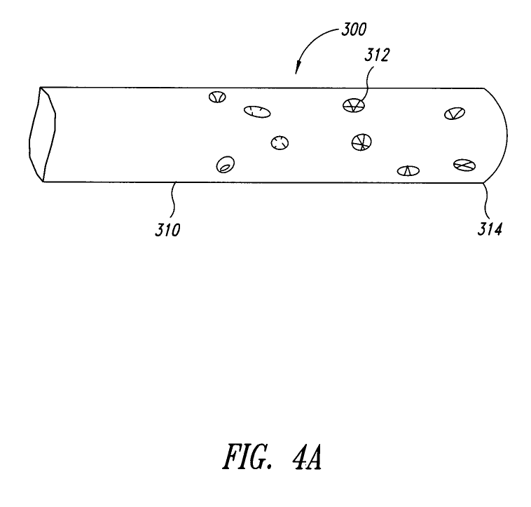

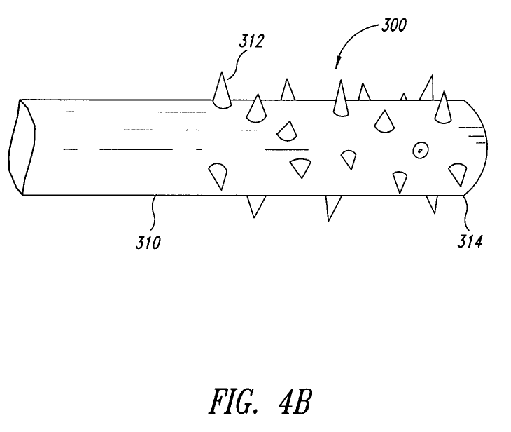

One or more markers can be positioned along the distal tip 110. FIG. 3C shows a tissue engaging element 140 having a single radio-opaque marker 213 at its distal tip. The radio-opaque marker 213 can also be at other locations. For example, the tissue engaging element 140 of FIG. 4D has a plurality of radio-opaque markers 213. The number and position of the radio-opaque markers can be selected based on the treatment to be performed.

The markers are preferably made from a material readily identified after insertion into a patient's body by using visualization techniques, such as the techniques noted above. If x-ray fluoroscopy is employed, the markers are preferably made from gold, tungsten, or any other readily identifiable material.

The distal tip 110 may carry one or more selectively deliverable medicaments that may or may not be related to the energy therapy to be performed. The medicaments (including eluting and/or non-eluting medicaments) may not be “activatable” treatment agents and, consequently, can function independently of the activatable treatment PDT agents. Medicaments can include, without limitation, radioactive materials (e.g., radioactive seeds), biologics, antibiotics, anti-inflammatory drugs, and the like. The medicaments may be impregnated into the balloon 139 (or other component of the distal tip 110) for a controlled slow release. In some embodiments, the balloon 139 can slowly drip or otherwise release the medicament into the patient.

To reduce or limit tissue growth, for example, the device 110 may include a passive growth inhibitor (e.g., a non-photosensitive growth inhibitor, proliferation inhibitors, vascular cell growth inhibitors, such as PDGF inhibitors, Trapidil, cytotoxin, and the like) to limit, minimize, or substantially prevent cell proliferation. The energy activated treatment agent can destroy tissue and the growth inhibitor can limit new tissue growth. In this manner, the treatment agent and growth inhibitor work in combination to effectively eliminate or limit unwanted tissue growth.

In other embodiments, the distal tip 110 may comprise one or more growth promoters that facilitate cell proliferation. The energy activated treatment agent can destroy unwanted abnormal tissue leaving substantially health, normal tissue. The growth promoter can then stimulate growth of this healthy tissue. A treatment program can include repeatedly destroying undesirable tissues and promoting growth of healthy tissue resulting in the rapid elimination of wanted tissue and reendothelialization of healthy, normal tissue.

FIGS. 4A and 4B show another embodiment of a distal tip that may be generally similar to the embodiments illustrated in FIGS. 1 to 3D, except as further detailed below. The expandable member 300 includes an array of deployable tissue engaging elements 312 positioned within an outer body 310. To deploy the inflatable elements 312, an inflation fluid can be delivered through the body 310 into the distal tip 314, through inflation ports, and into the elements 312 until the elements 312 are partially or fully distended, as illustrated in FIG. 4B. The body 310 can generally maintain its shape even during and/or after deployment of the tissue engaging elements 312. That is, the body 310 preferably does not distend during use. The material forming the body 310 is preferably strong enough to maintain its general shape during the inflation process but is preferably sufficiently flexible to allow navigation along tortuous delivery paths within the patient. Accordingly, the balloon 300 provides controlled localized inflation for targeting specific treatment regions.

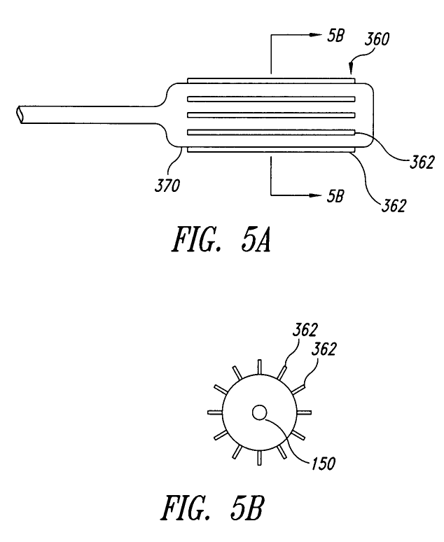

FIG. 5A shows another embodiment of a distal tip having a plurality of elongate deployable tissue engaging elements 362 in a fully deployed configuration. The elongate elements 362 extend longitudinally along at least a portion of a distal region 370 of the distal tip 360. The length of the elongate elements 362 can be selected based on any desired criteria, such as the length of the treatment site.

Although not illustrated, the tissue engaging elements of the distal tip can comprise one or more of the following: ribs (e.g., annular ribs), finger-like members, spikes, conical members (as discussed above), cylinders, and pyramids. Other configurations are also possible. Additionally the inflatable elements can be spaced evenly or unevenly along the body and are preferably positioned near a distal end of the catheter.

The deployable tissue engaging elements can have any suitable cross-section. For example, the elongate engaging elements can have a somewhat rectangular axial cross-section, polygonal axial cross-section, triangular axial cross-section, or any other suitable cross-section for engaging the cells at the target site. The elements 362 of FIG. 5B have a generally rectangular axial cross-section.

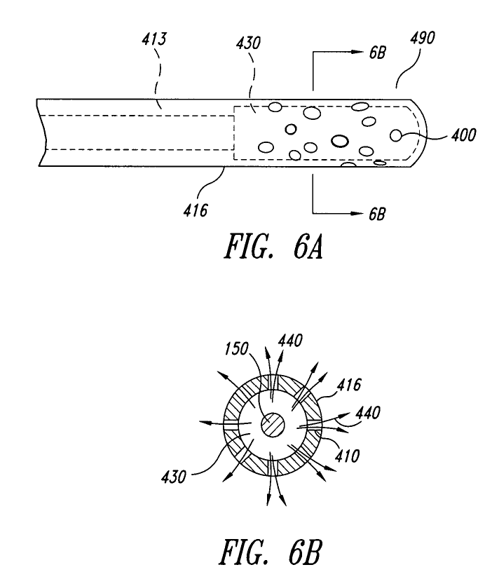

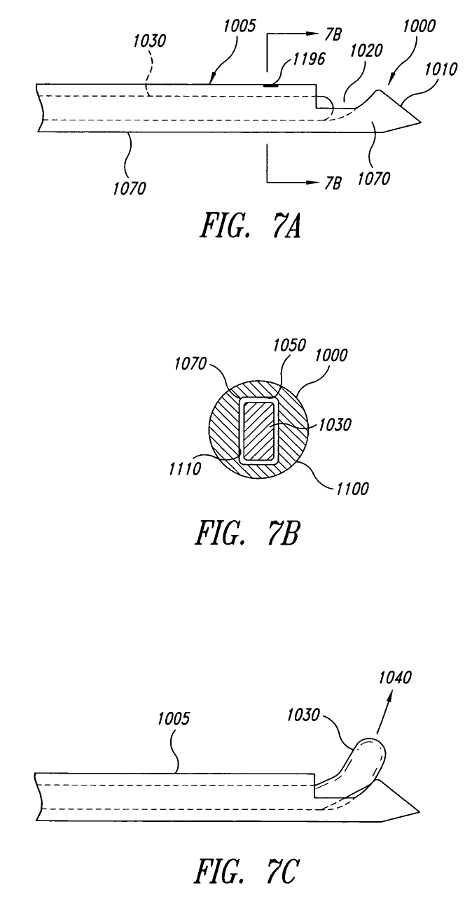

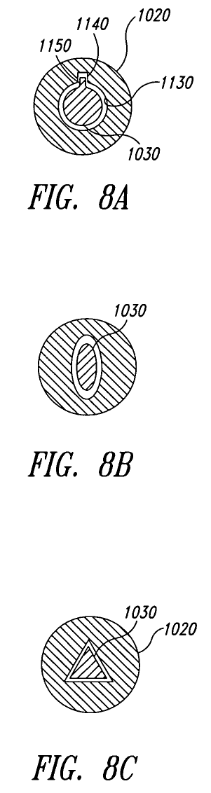

FIG. 6A shows an embodiment of a distal tip 490 without deployable tissue engaging elements. Distal tip 490 has an outer body 416 including one or more apertures 400 formed by through holes 410. The apertures 400 function as outlet ports for jetting out fluid. Fluid can be expelled from the distal tip 490 before, during, and/or after light is delivered out of the distal tip 490.

When the distal tip 490 is placed in situ, fluid is delivered along a lumen 413 extending through the catheter until it reaches a chamber 430 (shown in phantom in FIG. 6A) in the distal tip 490. The fluid then flows radially outward through the through holes 410, as indicated by the arrows 440, towards the patient's tissue. Similar to the tissue engaging elements discussed above, the jetting fluid can increase the effectiveness of the light therapy. Although not illustrated, valves, flow regulators, or other flow structures or devices can be utilized for selectively controlling fluid flow out of the distal tip 490. Fluid flow can thus be controlled to accurately deliver fluid at a desired flow rate and pressure to a specific target region.

In some embodiments, the array of apertures 400 generally surrounds at least one of the light sources 150 within the distal tip 490. When the light source 150 is energized, the fluid flowing out of the distal tip 490 acts as a light pathway introducer that defines paths for light travel. For example, the fluid flow can cause deformation or separation of tissue thereby defining light flow paths to specific cells of interest. To enhance performance, the light sources can direct light towards the apertures 400 so that the light travels along these well defined paths.

The fluid illustrated in FIG. 6B is preferably a substantially clear liquid. Saline or other biocompatible liquids can be employed. In some embodiments, gases (e.g., oxygen, ambient air, etc.) can be delivered out of the catheter. Gases are especially well suited for external applications or use in the respiratory system (e.g., the nasal cavity, bronchial passages, and the like). Various combinations of gases and liquids can be used based on the treatment.

The fluid can also contain one or more additives, such as treatment agents or medicaments including, without limitation, antibiotics, light sensitive agents, growth agents or inhibitors, and the like. If a light sensitive agent (e.g., a photo-reactive or photosensitive agent) is utilized, the light sources of the catheter preferably emit radiation wavelength(s) or waveband(s) that corresponds with, or at least overlap with, the wavelength(s) or waveband(s) that excite or otherwise activate the agent. Photosensitive agents can often have one or more absorption wavelengths or wavebands that excite them to produce substances which damage, destroy, or otherwise treat target tissue of the patient. Other types of drugs can be delivered to the patient as well.

FIG. 7A shows a delivery system 1000 for placing a light delivery device at a selected target site with the patient. Generally, a distal portion 1010 of an insertion device 1005 (illustrated as an insertion needle) can be transcutaneously advanced into the patient until a deployment window 1020 is at a desired location. During insertion of the device 1005, a light delivery device 1030 is preferably housed within the insertion device 1005, as shown in FIG. 7A. After placement of the insertion device 1005, the light delivery device 1030 is moved distally out the deployment window 1020, as indicated by the arrow 1040, for performing subcutaneous treatments.

In the illustrated embodiment of FIGS. 7A-7C, the light delivery device 1030 is in a working lumen 1050 (FIG. 7B) extending proximally from the window 1020 through the insertion device 1005. The light delivery device 1030 is preferably rotationally fixed with respect to the insertion device 1005. To position and steer the light delivery device 1030, the insertion device 1005 can be rotated to correspondingly rotate the light delivery device 1030. Once the light delivery device 1030 extends out of the window 1020 into the patient's tissue (see FIG. 7C), a torque can be applied to the light delivery device 1030 by rotating the insertion device 1005 about its longitudinal axis. In this manner, the light delivery device 1030 can be controllably steered for convenient delivery and aimed at target tissue. For example, a light delivery device 1030 may have a one-sided light bar that emits a field of light which can be accurately aimed at desired target site.

With continued reference to FIG. 7A, the insertion device 1000 is in the form of an insertion needle having the tapered distal portion 1010 for piercing tissue and a generally cylindrical outer body 1070. The outer body 1070 extends proximally from the distal portion 1010 and defines the window 1020.

The illustrated working lumen 1050 has an elongated cross-section which mates with a similarly shaped outer surface of the light delivery device 1030. The working lumen 1050 and light delivery device 1030 can have generally elliptical, polygonal (e.g., rectangular, square, triangular, etc.), oblong, or other suitable axial cross-sections to rotationally fix the light delivery device 1030 with respect to the insertion tool 1005 while allowing axial relative movement.

FIGS. 8A to 8C are axial cross-sectional views of light delivery devices disposed within insertion tools. FIG. 8A shows a working lumen 1130 having a slot 1140 that receives a protrusion 1150 of the light delivery device 1030. Other types of alignment structures, such as ribs, protrusions, pins, grooves, keyways, and the like can be used to prevent axial rotation of the light delivery device relative the insertion tool while allowing axial movement of the light delivery device 1030 through the insertion tool 1005. The light-generating apparatuses disclosed in U.S. Patent Publication No. 2005/0228260 can be modified for coupling to insertion tools or other delivery devices in a similar manner.

One or more flags, indicators, or other indicia can be used to help navigate the light delivery device 1030. The insertion tool 1005 of FIG. 7A has an indicator 1196 that can be used to determine the angular position of the tool 1005 and, thus, the position of the light delivery device 1030.

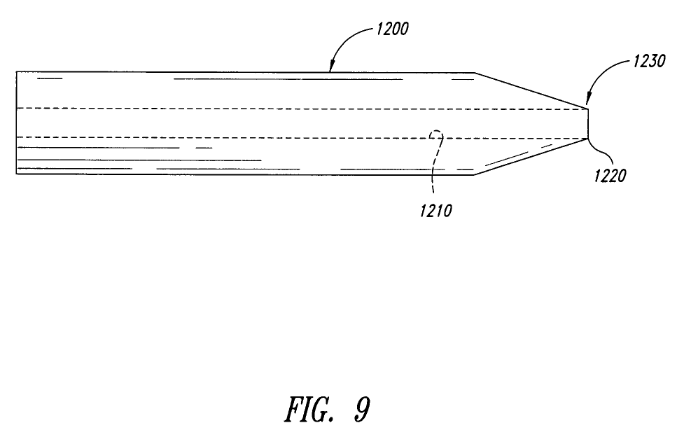

FIG. 9 shows another embodiment of an insertion device 1200 that is similar to the insertion tool 1005, except as detailed below. The insertion device 1200 has generally centrally disposed working lumen 1210. The working lumen 1210 defines an inner portion of a cutting edge 1220 for cutting through tissue. Advantageously, a light delivery device can be advanced axially out the distal end 1230 of the device 1200.

In other embodiments, the insertion device can be in the form of a delivery sheath, introducer, trocar, or endoscopic instrument. Each of these insertion devices can have a working lumen dimensioned to receive the light delivery devices described herein (or in the references incorporated by reference) while inhibiting or preventing rotational movement of the light delivery device relative to the insertion device.

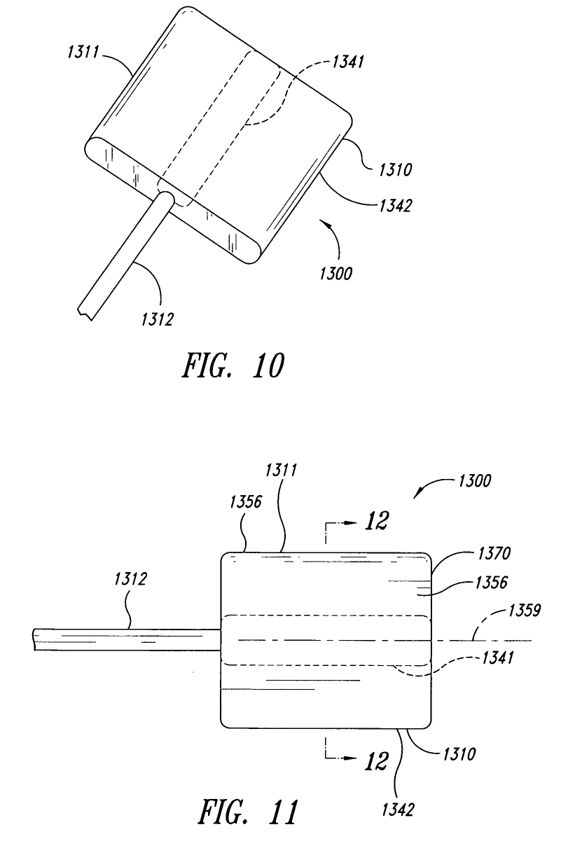

FIGS. 10 to 12 show a light delivery apparatus 1300 suitable for treating relatively large target areas. The light delivery apparatus 1300 includes an expandable distal tip 1310 and a catheter body 1312 extending proximally from the distal tip 1310. The somewhat flat distal tip 1310 for producing a wide light field is suitable for external and internal light therapy. Advantageously, the distal tip 1310 can emit a generally uniform field of a therapeutic amount of light for a somewhat homogenous treatment of the target tissue.

The distal tip 1310 includes a controllably expandable member 1311 surrounding an internal light source 1316 positioned to emit light that passes through the member 1311. The illustrated light source 1316 is a one-sided light bar disposed within a chamber 1320 defined by an inner surface 1322 of the distal tip 1310. A selectively movable reflector 1330 for reflecting light emitted from the light source 1316 is coupled to the inner surface 1322 of the expandable member 1311. The reflector 1330 can reflect light rays 1334 emitted from the activated light source 1316 in a desired direction. The reflected light rays 1334 can then pass through the chamber 1320 and a transmissive portion 1340, resulting in light rays being emitted from one side of the distal tip 1310. Accordingly, the distal tip 1310 can redirect light to provide a high intensity light field.

The illustrated expandable member 1311 is in the form of an inflatable balloon movable between a collapsed configuration 1341 (shown in phantom in FIGS. 10 and 11) and an expanded configuration 1342. The expandable member 1311 occupies first and second volumes when in the collapsed and expanded configurations 1341, 1342, respectively. The second volume is preferably greater than the first volume. The level of inflation can vary depending on physiological features of the subject and the light therapy to be performed. The physiological features, for example, can be optical characteristics of the target site, available space to accommodate the distal tip 1310, and the like. The amount of inflation of the expandable member 1311 can be increased or decreased to increase or decrease the spreading of the light.

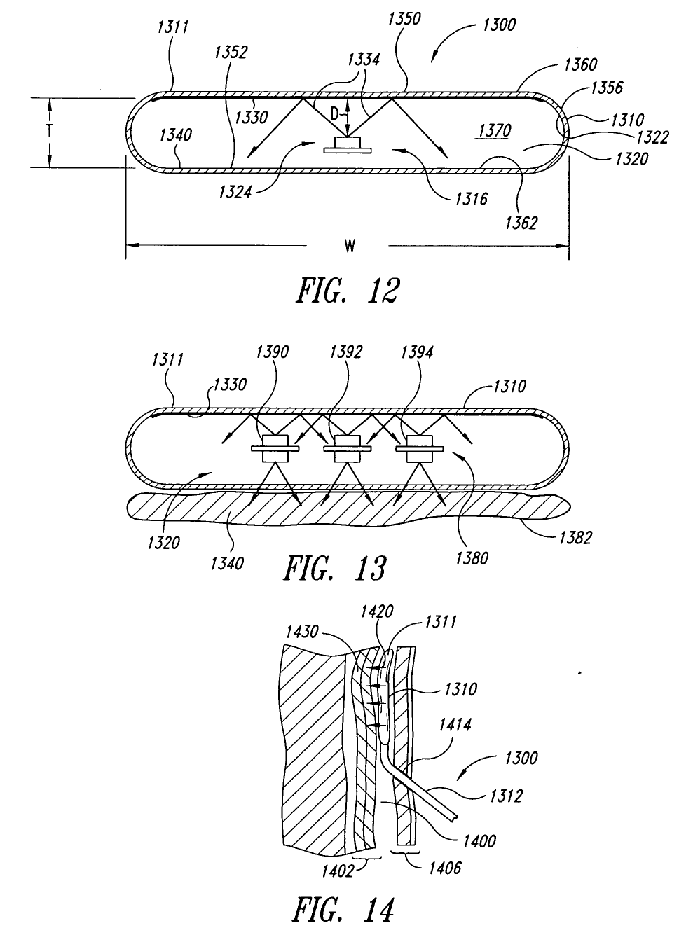

The expandable member 1311 can be moved between the collapsed configuration 1341 and expanded configuration 1342 to adjust the distance D (FIG. 12) between the light source 1316 and the reflector 1330, angle of incidence, angle of reflectance, and the like to achieve the desired light distribution, light field intensity, and the like.

Referring to FIG. 12, the expandable member 1311 includes a first portion 1350, a second portion 1352, and a sidewall 1356 extending between the first and second portions 1350, 1352. The first and second portions 1350, 1352 are flexible sheet-like members that can closely surround the light source 1316 when the expandable member 1311 is collapsed.

The illustrated sidewall 1356 extends between the outer edges 1360, 1362 of the first and second portions 1350, 1352, respectively. The first portion 1350, second portion 1352, and sidewall 1356 cooperate to define the chamber 1320 suitable for holding a flowable substance (e.g., an inflation fluid). The illustrated sidewall 1356 is curved outwardly (illustrated as a convex sidewall), but other configurations are also possible.

The expandable member 1311 of FIG. 12 has a width W that is greater than its thickness T. The width W can be at least 2 times, 3 times, 4 times, or 5 times greater than the thickness T. In some embodiments, the width W is at least 3 times greater than the thickness T to form a generally flat expandable member 1311. However, if a flat expandable member is not needed or desired, the expandable member 1311 can have a circular axial cross-section, elliptical axial cross-section, or other cross-sections based on, for example, any spaces or cavities in the subject suitable for receiving and allowing expansion of the member 1311.

The light source 1316 can be interposed between the reflector 1330 and the transmissive portion 1340. Both the reflector 1330 and transmissive portion 1340 are movable relative to the light source 1316. When the expandable member 1311 is inflated (either partially or fully inflated), the light source 1316 can be somewhat centrally disposed in the chamber 1320 for an enhanced light distribution.

The illustrated light source 1316 can extend longitudinally along a longitudinal axis 1359 of the distal tip 1310. For example, the light source 1316 can extend from the catheter body 1312 to a distal section 1370 of the sidewall 1356. In other embodiments, the light source 1316 is offset from the longitudinal axis 1359 of the distal tip 1310. To maintain proper positioning of the light source 1316 with respect to the member 1311, one or more tethers, spaces, or other positioning means can extend between the member 1311 and the light source 1316.

To limit or substantially prevent direct illumination of untargeted tissue (e.g., tissue contacting the first portion 1350), the reflector 1330 can inhibit or prevent the transmission of light therethrough. In some embodiments, the reflector 1330 can reflect at least a substantial portion of the incident light striking it, thus minimizing collateral damage to untargeted tissue. In other embodiments, the reflector 1330 may transmit a portion of the light incident thereon and reflect the other portion of the light incident thereon.

The reflectivity of the reflector 1330 can be increased or decreased to increase or decrease the intensity of the light field. Various types of high, medium, and low reflectance reflectors can be used based on the light therapy to be performed. In some non-limiting embodiments, the reflector 1330 has a reflective index of at least about 60%. Thus, the reflector 1330 reflects about 60% of the light energy striking it. The other portion of the light energy can be absorbed or transmitted. In some non-limiting embodiments, the reflector 1330 has a reflectivity index of at least about 70%, 80%, 90%, or ranges encompassing such indexes. In some high reflectance embodiments, the reflector 1330 can reflect most or substantially all incident light thereon.

If a somewhat uniform light distribution is desired, the reflector 1330 can be a diffuse reflector that scatters light in numerous directions. In other embodiments, the reflector 1330 provides specular reflection. Such a reflector can be generally a smooth mirror (e.g., a somewhat rigid mirror or a flexible mirror) having minimal surface irregularities. In some highly reflective embodiments, the reflector 1330 is a reflective film formed of reflective mylar, metals (e.g., aluminum), reflective polymers, or other materials with a high reflectivity. One of ordinary skill in the art can select the reflective materials based on desired physical properties, optical properties, and thermal properties. For example, flexible reflective materials can be used to form a distal tip capable of conforming to highly contoured surfaces and moving readily between the collapsed and expanded configurations.

In operation, the distal tip 1310 can be in the collapsed configuration 1341 for delivery through a working lumen of an insertion device. The collapsed distal tip 1310 can be folded-up (similar to the distal tip 110 in FIG. 1), rolled up, or another state such that it occupies a smaller volume as compared to its expanded state. After the insertion device is placed in the subject, the collapsed distal tip 1310 can be advanced into the subject and, once positioned, inflated a desired amount.

The size and shape of the distal tip 1310 can be chosen based on the size and shape of the targeted tissue. If the distal tip 1310 is in proximity to or contacts the targeted tissue during light therapy, the reflector 1330 can generally match the shape and size of the target tissue. In such embodiments, the reflector 1330 can help illuminate a substantial portion of the targeted tissue.

If the distal tip 1310 is spaced away from the targeted tissue, the reflector 1330 can be smaller than the targeted tissue. The reflector 1330 can help distribute the light outwardly and, consequently, can be significantly smaller than the target site, especially when spaced a significant distance from the target site.

In some embodiments, the expandable member 1311 is made, in whole or in part, of an optically transparent material that permits light emitted from the light source 1316 from passing therethrough. In some embodiments, the expandable member 1311 comprises an opaque material that inhibits or substantially prevents light emitted from the light source 1316 from passing therethrough. For example, the reflector 1330 of FIG. 12 can be replaced with an opaque material such that the distal tip 1310 emits light from one side. Thus, opaque materials can block light to limit or prevent light therapy on untargeted tissues. Various combinations of reflectors and opaque materials can be used to produce the desired light field.

FIG. 13 shows a two-sided light source 1380 for emitting light towards the reflector 1330 and the transmissive portion 1340. Most or a substantial portion of the light from the light source 1380 can be directed towards the target tissue 1382. The illustrated light source 1380 comprises a plurality of light bars 1390, 1392, and 1394 spaced laterally from one another. Any number of light bars can be disposed within the chamber 1320 to achieve the desired light distribution.

FIG. 14 shows the distal tip 1310 positioned within an internal space or cavity 1400 between an omentum 1402 and an abdominal wall 1406. To deliver the distal tip 1310 to the cavity 1400, the collapsed inflatable member 1311 is passed through an insertion device (e.g., an insertion needle, introducer, trocar, etc.). The insertion device can form a hole or passageway 1414 through abdominal wall 1406, thereby providing access to the cavity 1400. Once the inflatable member 1311 is positioned in the cavity 1400, an inflation medium can flow through the catheter body 1312 into the chamber 1320 until the inflatable member 1311 has been expanded a desired amount.

When activated, the light source 1316 emits light rays 1420 towards the omentum 1402. This light 1420 can interact with one or more treatment agents to photo-activate or photo-excite target tissue 1430 (e.g., intra-abdominal fat) in or around the omentum 1402. The light can therefore cause localized cell destruction, size reduction, and/or necrosis of intra-abdominal fat resulting in weight loss. The distal tip 1310 can be navigated to various locations in the cavity 1400 in order to perform light therapy on different target sites.

Because the expandable member 1311 is compliant, it can conform to the shape of the cavity 1400 or adjacent tissue. If the target tissue 1430 has a complex shaped outer surface, the expandable member 1311 can be sufficiently compliant to conform closely to the outer surface resulting in efficient light transmission to the targeted tissue. In some embodiments, for example, the expandable member 1311 can overlay, cover, or otherwise drape over an internal organ to facilitate light delivery to that organ or remote tissue. To perform light therapy on the serous membrane, for example, the expandable member 1311 can drape over the bowel to deliver light to the serosal surfaces. The light can also be delivered into the recesses of the abdomen or thorax, as well as other somewhat hard to reach tissue.

With continued reference to FIG. 14, intra-abdominal fat, including mesenteric adipose tissue and omental adipose tissue, can be located in the peritoneal cavity. This tissue may contribute to the onset of diseases, including metabolic disorders (e.g., arteriosclerosis, diabetes, and/or hyperlipemia) and cardiac vessel disorders. The emitted light 1420 can destroy, reduce in size, or otherwise treat the adipose tissue such that these diseases can be reversed or otherwise improved.

Advantageously, the light delivery apparatus 1300 can treat adipose tissue without traumatizing tissue proximate the adipose tissue (e.g., the abdominal wall 1402), thus minimizing trauma to the patient. Because the expandable member 1311 has a generally flat shape, it can be expanded in situ while keeping trauma to the subject at or below a desired level. If the expandable member 1311 is expanded in the space 1400, it can help separate the omentum 1402 and the abdominal wall 1406 to further reduce the amount of light reaching the abdominal wall 1406. The distal tip 1310 can be used in other anatomical cavities to treat tissue in a similar manner.

With continued reference to FIG. 14, the light delivery apparatus 1300 can emit light while the expandable member 1311 rests against the target tissue. A relatively large treatment area of the omentum 1402 can be illuminated without illuminating collateral tissue. In the illustrated embodiment, the first portion 1350 faces the abdominal wall 1406 such that the reflector 1330 reflects light away from the abdominal wall 1406, thus protecting the abdominal wall 1406 from inadvertent light therapy. While some emitted light, in some instances, may be reflected through the cavity 1400 to collateral tissue, the amount of activated treatment agent and light in non-targeted tissue can be kept at or below an acceptable level.

The target tissue 1430 may be subjected to at least one wavelength of light that is approximately close to, if not the equivalent to, at least one excitation wavelength of the target tissue, according to some embodiments. It is understood that even if one cell is targeted, it is possible that other cells in the vicinity of the targeted cell may also be subjected to light emitted from the one or more light sources. It is contemplated that the expandable member 1311 can be expanded or contracted during, before, and/or after the light source 1316 is activated. Thus, the light field can be adjusted by altering the configuration of the expandable member 1311.

After performing light therapy, the expandable member 1311 can be deflated for subsequent removal from the patient. The light delivery apparatus 1300 can be used to perform another procedure or discarded. In other embodiments, the expandable member 1311 can be left in situ for subsequent light therapy procedures at the same site, or a nearby site, at a later point in time. Any number of light therapy procedures can be performed with a single expandable member 1311.

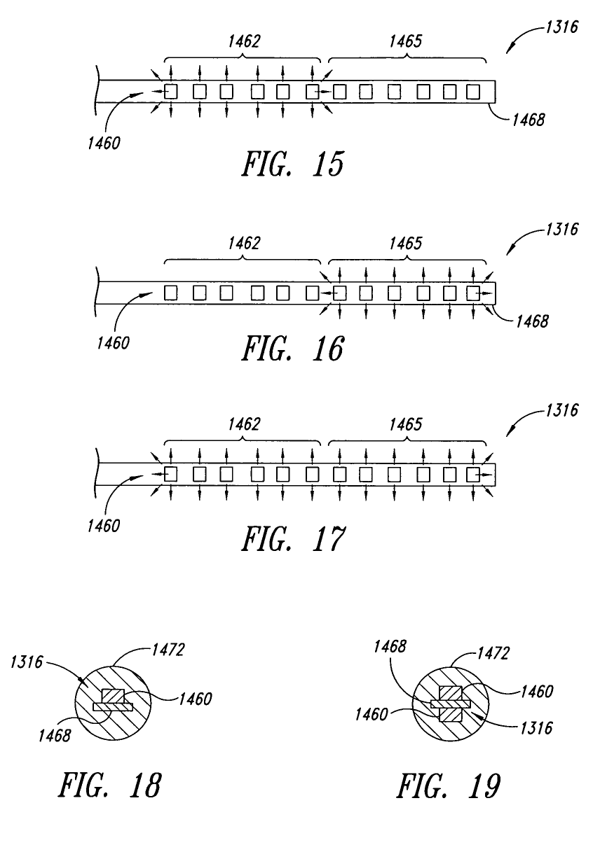

FIGS. 15 to 17 show the light source 1316 in the form of a light bar with independently activatable light emitting devices 1460 coupled to a substrate 1468. In the illustrated embodiment, the light source 1316 has a first set of light emitting devices 1462 and a second set of light emitting devices 1465. The first and second sets 1462, 1465 can be activated sequentially or concurrently. In FIG. 15, the first set of light emitting devices 1462 is energized. In FIG. 16, the second set of light emitting devices 1465 is energized. In FIG. 17, both the first and second sets 1462, 1465 are energized. The first and second sets 1462, 1465 can be independently operated to target different treatment sites.

The number, type, and spacing of the light emitting devices can be selected based on the desired light field to be generated. For example, each of the first and second sets 1462, 1465 of FIGS. 15 to 17 includes six light emitting devices 1460, but a lesser or greater number of light emitting devices can also be used.

The light source 1316 can be a one-sided or two-sided light bar. FIG. 18 shows the light source 1316 in the form of a one-sided light bar encapsulated in a protective covering 1472 (illustrated as a cylindrical covering). FIG. 19 shows the light source 1316 in the form of a two-sided light bar.

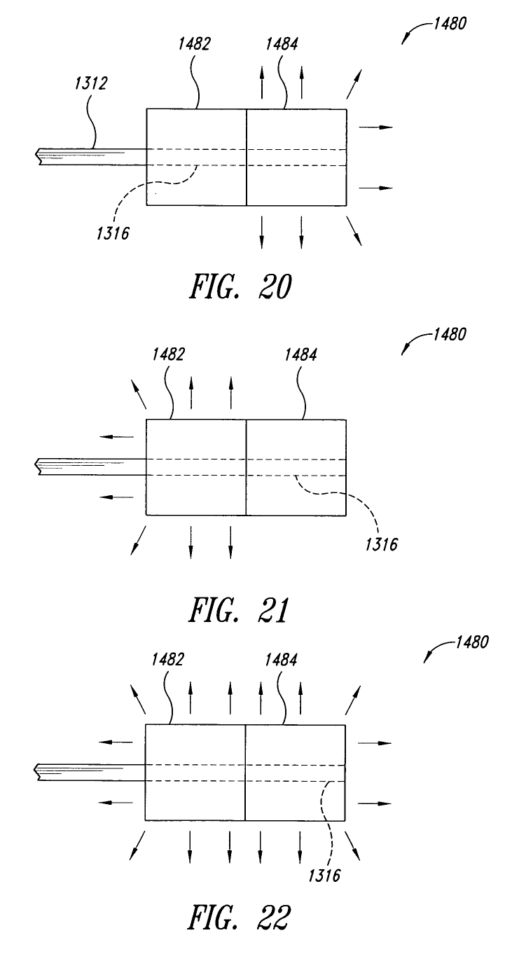

FIGS. 20 to 22 show an expandable assembly 1480 surrounding the light source 1316 of FIGS. 15 to 17. The illustrated expandable assembly 1480 includes first and second expandable members 1482, 1484 (both illustrated in an expanded configuration) surrounding the first and second sets 1462, 1465, respectively. Each of the independently inflatable expandable members 1482, 1484 may be generally similar to the expandable member 1311 illustrated in FIGS. 15 to 17.

The expandable assembly 1480 can have any number of expandable members arranged linearly, side-by-side, or in any other suitable arrangement. The separately operable expandable members, each containing one or more groups of activatable light emitting sources, can be inflated sequentially or concurrently.

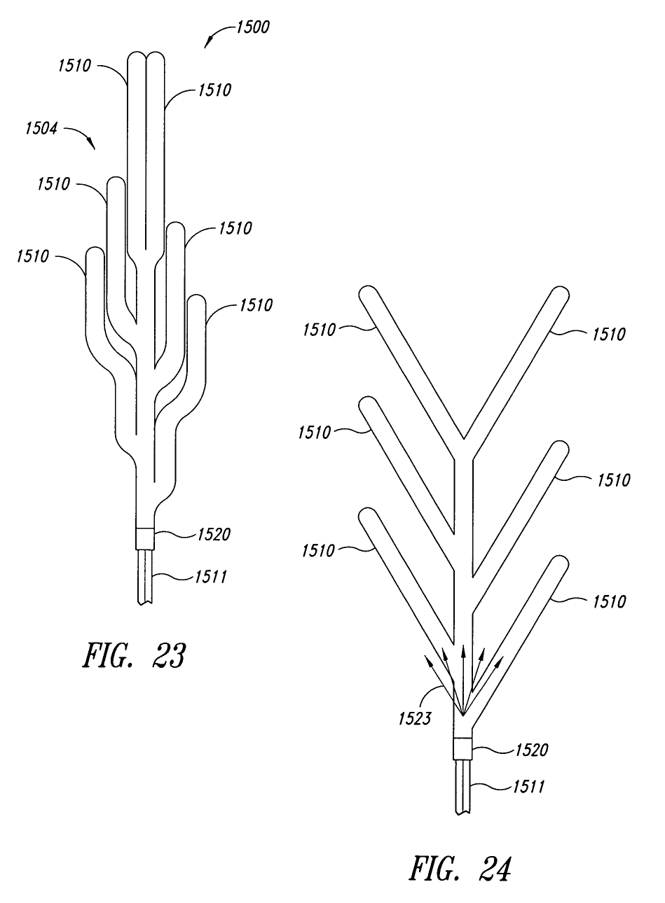

FIGS. 23 and 24 show a light delivery apparatus 1500 having an expandable distal tip 1504 operable to facilitate delivery of light to internal tissue. The expandable distal tip 1504 includes a plurality of selectively deployable fingers 1510 movable between a low-profile configuration (FIG. 23) and an expanded configuration (FIG. 24). A catheter body 1511 extends proximally from the distal tip 1504. A light source 1520 is disposed along the catheter body 1511. When activated, the light source 1520 emits light 1523 to illuminate tissue in proximity to the tip 1504.

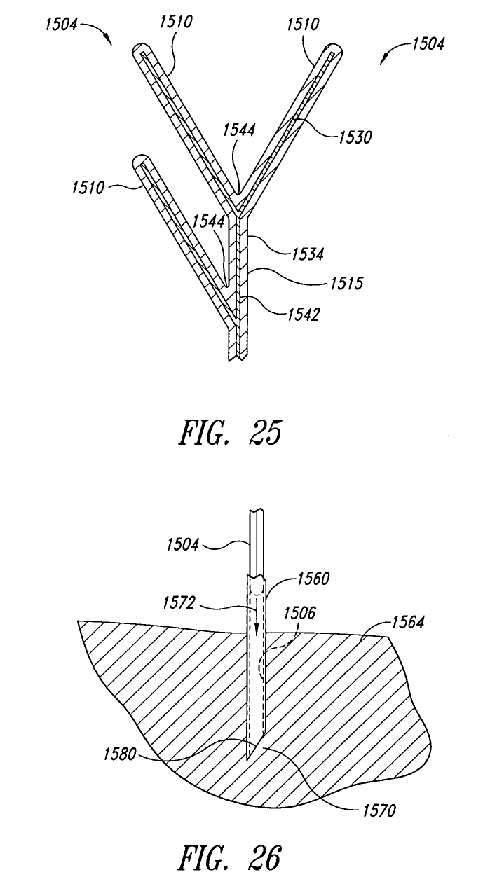

Referring to FIG. 25, the expandable distal tip 1504 can have a frame 1530 embedded in an outer covering 1534. The frame 1530 can have a selected or preset configuration generally corresponding to the desired expanded configuration. In some embodiments, the selected configuration can be within a range of configurations. While the distal tip 1504 in the selected configuration may vary in shape depending on externally applied forces, the distal tip 1504 can bias towards certain configuration.

In self-expanding embodiments, the frame 1530 can comprise one or more shape memory materials, which can move the tip 1504 between one or more configurations when activated. The illustrated distal tip 1504 can be moved from the collapsed configuration to the expanded configuration in response to thermal activation. The shape memory material may include, for example, a shape memory alloy (e.g., NiTi), a shape memory polymer, a ferromagnetic material, or other material. The shape memory material can be activated by an external energy source (e.g., an ultrasound energy source, heaters, and the like), internal heating elements, and the like.

The covering 1534 can be coupled to the frame 1530 to prevent relative movement therebetween. The covering 1534 can be formed by an overmolding process, spray coating process, dip coating process, or other deposition process suitable for forming a layer of material. Bonding processes, adhesion processes, or fusion processes can fixedly couple the cover 1534 to the frame 1530.

The covering 1534 can facilitate light delivery and, thus, may comprise a light guide material. Light guide material can help transmit light from the light source 1520 towards the targeted tissue. Exemplary light guide materials can include, without limitation, waveguide polymers, optical plastics, optical polymers (e.g., poly(methyl methacrylate), acrylics, elastomeric polymers), and other types of materials the can help guide light for enhanced light penetration. In some embodiments, one or more optical fibers, fiber bundles, cables, liquid light guides, or other types of waveguide or light guides can transmit light to the desired location. These optical components can be incorporated into the covering 1534, frame 1530, or both. Optical fibers can have a core, jacket, cladding, and other structures that are known in the art to obtain the desired optical properties, such as attenuation, bandwidth, dispersion, mechanical properties (e.g., tensile strength, flexibility, etc.), and the like.

Various types of connections can selectively articulate the fingers 1510. In some embodiments, articulatable junctions 1544 can rotatably connect respective fingers 1510 to a central frame body 1542. After the collapsed distal tip 1504 is positioned within the patient, the junctions 1544 are activated to move to a preset or memorized shape to bias the fingers 1510 toward the expanded configuration.

Referring to FIGS. 23 to 25, the fingers 1510 can be evenly or unevenly spaced longitudinally along the length of the expandable distal tip 1504. Alternatively or additionally, the fingers 1510 can be evenly or unevenly spaced circumferentially about the tip 1504. In some embodiments, a plurality of fingers 1510 are positioned on one side of the tip 1504. In other embodiments, a plurality of fingers 1510 are positioned on opposing sides of the distal tip 1504. However, other configurations are also possible.

FIGS. 26 to 28 show one embodiment of delivering and deploying a light delivery apparatus. In FIG. 26, an insertion device 1560 is inserted into tissue 1564 of the patient. Once inserted, the distal tip 1504 of the light delivery apparatus 1500 is advanced through a working lumen 1506 of the insertion device 1560 towards the target site 1570, as indicated by the arrow 1572. Because the distal tip 1504 can be in a low-profile configuration, a correspondingly low-profile insertion device 1560 can be employed to limit trauma to the subject.

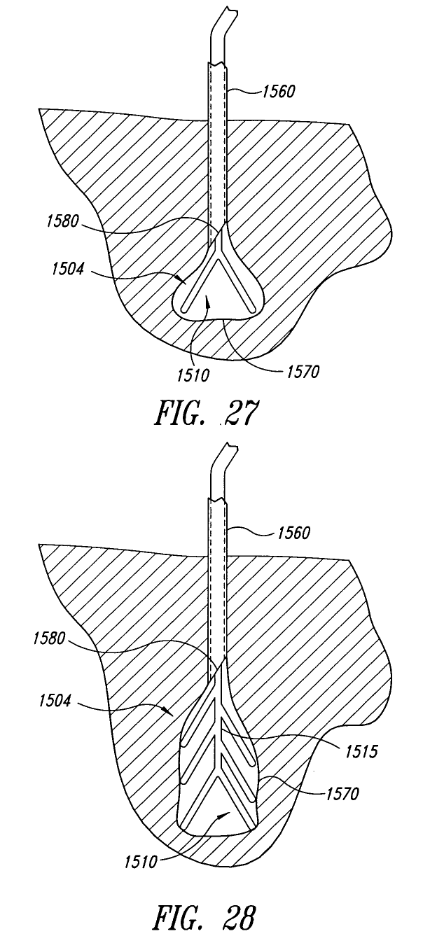

In some self-expanding embodiments, the fingers 1510 are biased radially outward, as noted above. The fingers 1510 can be radially restrained by the insertion device 1560 to limit self-expansion. The wall of the insertion device 1560, for example, can restrain the fingers 1510 until the distal tip 1504 is delivered out of the insertion device 1560. Once released, each finger 1510 is rotate outwardly towards its expanded configuration, as shown in FIGS. 27 and 28. Accordingly, the distal tip 1504 can self-expand under its own bias to its enlarged cross-section.

The expansion of the tip 1504 can increase the illuminated surface area of targeted tissue 1570 for enhanced light therapy procedure. Additionally or alternatively, the tip 1504 can create or enlarge a cavity in the tissue 1564. The configuration of the tip 1504 can be selected based on the desired biasing forces suitable for creating cavities, enlarging existing cavities, displacing tissue, or damaging tissue (e.g., tearing tissue, puncturing tissue, and the like).

In some embodiments, the distal tip 1504 can be advanced distally out of the end 1580 of the generally stationary insertion device 1560. As such, the end 1580 can be positioned proximally of the target site 1570, as shown in FIG. 26. The distal tip 1504 is slid distally through the insertion device 1560 and is then passed through any intermediate tissue until it reaches the target site 1570.

In other embodiments, the distal tip 1504 is generally stationary with respect to the subject while the insertion device 1560 is pulled proximally so as to unsheathe the distal tip 1504. Once the fingers 1510 are exposed, the distal tip 1504 may be moved (e.g., pulled proximally, pushed distally, or both) to further open up the distal tip 1504, displace tissue, and the like.

As shown in FIG. 28, the expanded distal tip 1504 can occupy a larger volume than the same distal tip 1504 in its low profile configuration. As such, the outwardly biasing fingers 1510 can form an enlarged cavity. The light source of the distal tip 1504 can then be activated to illuminate the target tissue 1570.

Even though the fingers 1510 can be brought inwardly to tightly surround the central main body 1515, the fingers 1510 can exert a sufficient biasing force to penetrate, displace, or otherwise physically interact with tissue while limiting unwanted trauma. The fingers 1510, for example, can atraumatically apply a desired amount of outwardly directed pressure to surrounding tissue. If the distal tip 1504 is used to perform phototherapy on adipose tissue, the fingers 1510 may be capable of penetrating the adipose tissue without damaging other types of tissues, thereby reducing the likelihood of damage to other organs, such as blood vessels, bowels, or other viscera or internal organs. The tips of the fingers 510, for example, may be slightly blunt to provide an atraumatic surface for engaging certain types of tissue.

In other embodiments, the fingers 1510 can be deployed outwardly to cut, slice, tear, or damage the tissue as part of a treatment procedure. The edges of the fingers can be serrated or sharpened to cut and slice tissue. In some embodiments, the fingers 1510 can apply sufficiently large forces to tear tissue. The tips of the fingers 1510 can also be sharpened to limit slipping or to pierce tissue. Tissue damaged can increase the light penetration depth to enhance treatment performance, although healing time may also be increased.

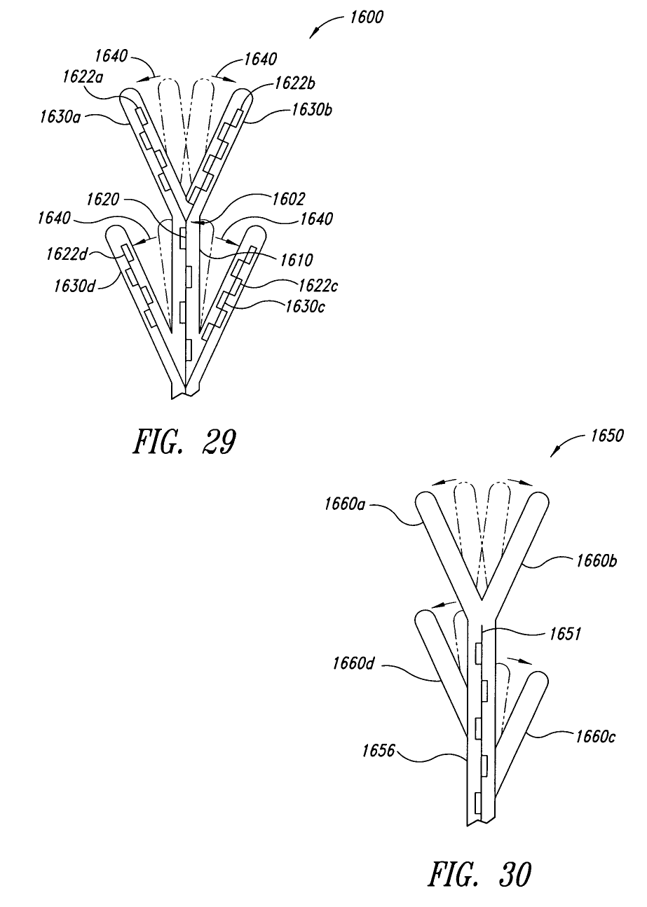

FIGS. 29 and 30 show distal tips for performing light therapy that may be generally similar to the distal tip 1504 illustrated in FIGS. 23 to 25, except as further detailed below.

The distal tip 1600 of FIG. 27 includes a light source 1602 in the form of a light bar extending through a central main body 1610. The illustrated light source 1602 includes a central light bar 1620 and electrically connected peripheral light bars 1622a-d extending through corresponding fingers 1630a-d. Advantageously, the peripheral light bars 1622a-d can be in proximity to targeted tissue adjacent the fingers 1630a-d for efficient light delivery. The illustrated fingers 1630a-d and respective peripheral light bars 1622a-d can be moved from the collapsed configuration (shown in phantom) to the expanded configuration, as indicated by the arrows 1640.

The distal tip 1650 of FIG. 30 has a light source 1651 in the form a single light bar extending axially through a central main body 1656. The fingers 1660a-d can serve as movable light guides for directing light outwardly from the light source 1651.

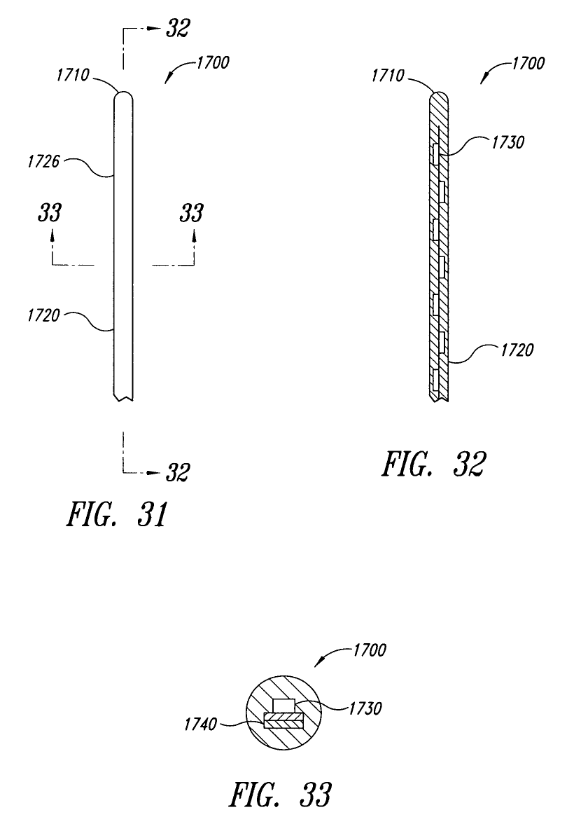

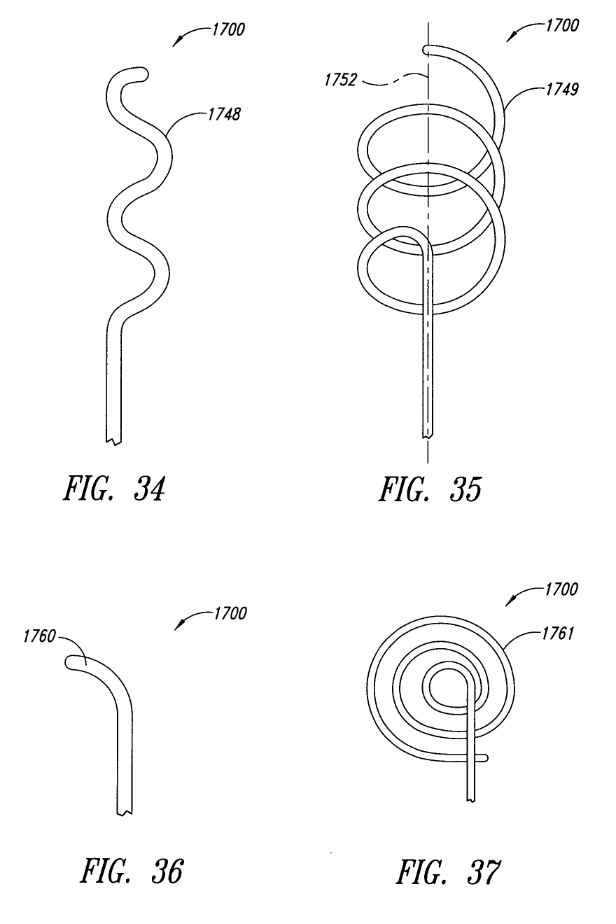

FIGS. 31 to 33 show a generally straight elongate tip 1700 movable between a first configuration (illustrated as a generally straight configuration) to a second configuration, such as those illustrated in FIGS. 34 to 37. The tip 1700 in the first configuration can treat a first area or volume of tissue, wherein the tip 1700 in the second configuration can treat a second area or volume of tissue that is greater than the first area or volume tissue. That is, the tip 1700 in the second configuration can illuminate a greater volume of tissue as compared to when it is in the first configuration.

With continued reference to FIG. 31, the elongate tip 1700 includes a distal end 1710, a proximal end 1720, and an elongate tip main body 1726 extending between the distal and proximal ends 1710, 1720. As shown in FIG. 32, a light source 1730 extends axially along the length of the main body 1726. The proximal end 1720 can be coupled to a catheter body, through which power is delivered to the light source 1730.

The illustrated elongate tip 1700 of FIG. 33 includes an activatable frame 1740 having a preset configuration corresponding to the second configuration. The frame 1740 may comprise a shape memory material that biases, when activated, from the initial straight configuration of FIGS. 31 and 32 to the second configuration. Additionally, the frame 1740 may have a plurality of individually activatable members, each having a different preset configuration and a different activation temperature. The frame 1740 can therefore have more than one preset configuration to move the tip 1700 between any number of positions as desired.

The tip main body 1726 can be formed of an optically transparent material for transmitting light delivered from the light system 1730. Similar to the distal tips described above, optical plastic can be used to assist in light distribution. Because the main body 1726 moves a significant distance, it can be formed of a relatively flexible material.

In some embodiments, the elongate tip 1700 can be formed in a selected or preset configurations. Plastics, polymers, elastomeric materials, and other formable materials can be molded and set in a mold cavity having a shape corresponding to the desired configuration (see FIGS. 34 to 37). When unrestrained, the elongate tip 1700 thus biases towards the selected configuration without performing a separate activation procedure.

The elongate tip 1700 can be configured for convenient navigation through and around tissue. The preset configuration can be chosen once a desired treatment site has been identified. FIGS. 34 to 37 illustrate exemplary shapes but other shapes can be used.

FIG. 34 shows the elongate tip 1700 having a serpentine configuration 1748.

FIG. 35 shows the elongate tip 1700 having a helix configuration 1749 (e.g., a somewhat corkscrew shape). A user can rotate the tip 1700 to move it distally or proximally through tissue. For example, the tip 1700 can be rotated about an axis of rotation 1752 (e.g., its longitudinal axis) to move the tip 1700 distally through tissue. The tip 1700 can be rotated in the opposite direction to move the tip 1700 proximally.

The elongate tip of FIG. 36 has a curved section 1760 for convenient retraction, rotation, and advancement. For example, to treat a circular volume, the tip 1700 can be sequentially retracted, rotated, advanced distally, and activated to deliver light. This process can be used to bring the target tissue within range of the light source in the tip 1700.

FIG. 37 shows the elongate tip 1700 having a spiral configuration 1761 for expanding outwardly. To perform light therapy between layers of tissue, the elongate tip 1700 can be inserted between two adjacent layers and then expanded to the illustrated configuration 1761. A relatively uniform light field can be generated with the expanded tip 1700.

In yet other embodiments, the distal tip 1700 can have, without limitation, an S-shape, C-shape, W-shape, V-shape, or other shapes that can generally correspond to the shape of the target site, thus focusing the light therapy on the targeted tissue.

To place the elongate tip 1700 in a subject, it can be inserted through an introducer, such as a straight insertion device. The introducer may be rigid, semi-rigid, or somewhat flexible. For example, the introducer can be in the form of a flexible delivery catheter or sheath. Once the tip 1700 is moved out of the introducer, an activation process can be performed to activate a shape memory material of the tip 1700. The activated shape memory material then biases towards its memorized configuration.

If the tip 1700 is formed with an initial preset expanded configuration, the introducer can be sufficiently rigid to hold the tip 1700 in a desired low-profile delivery configuration. As the elongate tip 1700 passes out of the introducer, it can bias towards its preformed configuration.

In both the activatable and preformed elongate tip embodiments, the outwardly expanding tip 1700 delivers light energy to a greater volume of tissue. A user can navigate the tip 1700 through and between internal tissues. Before, during, and/or after this navigation process, the light source can be activated to delivery light energy to the internal tissues.

Various access techniques can be used to deliver the light systems disclosed herein. Open procedures, semi-open procedures, laparoscopic procedures, endocscopic procedures, and minimally invasive procedures (e.g., percutaneous techniques) can provide suitable access to the target delivery site. Known conventional surgical instruments (e.g., sizing rings, balloons, calipers, gauges, delivery sheaths, catheters, tubes, cannulas, and the like) can be used to access the deployment sites. Many times, the access techniques and procedures can be performed by the surgeon and/or a robotic device, such as robotic systems used for performing minimally invasive surgery. Those skilled in the art recognize that there are many different ways to access internal deployment sites.

Target sites can include, without limitation, fat deposits (e.g., abdominal fat, subcutaneous fat, sub-mental fat, and the like), cancerous tissue, or other unwanted tissue. The light systems can be implanted in the targeted tissue for direct illumination, placed onto the target tissue for transillumination, or spaced from the target tissue.

All of the above U.S. patents, U.S. patent application publications, U.S. patent applications, foreign patents, foreign patent applications and non-patent publications referred to in this specification and/or listed in the Application Data Sheet, to include U.S. Pat. Nos. 4,675,338; RE 37180; 6,958,498; 6,784,460; 6,661,167; 5,800,478, and 6,445,011; U.S. Publication Nos. 2005/0228260; 2005/0085455A1; International Patent Application Nos. PCT/US2005/032851 and PCT/US01/44046; and U.S. patent application Ser. No. 10/687,579 are incorporated herein by reference, in their entirety. Except as described herein, the embodiments, features, systems, devices, materials, methods and techniques described herein may, in some embodiments, be similar to any one or more of the embodiments, features, systems, devices, materials, methods and techniques described in the incorporated references. In addition, the embodiments, features, systems, devices, materials, methods and techniques described herein may, in certain embodiments, be applied to or used in connection with any one or more of the embodiments, features, systems, devices, materials, methods and techniques disclosed in the above-mentioned incorporated references.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments disclosed herein. Similarly, the various features and steps discussed above, as well as other known equivalents for each such feature or step, can be mixed and matched by one of ordinary skill in this art to perform methods in accordance with principles described herein. Additionally, the methods which are described and illustrated herein are not limited to the exact sequence of acts described, nor are they necessarily limited to the practice of all of the acts set forth. Other sequences of events or acts, or less than all of the events, or simultaneous occurrence of the events, may be utilized in practicing the embodiments of the invention.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. The materials, methods, ranges, and embodiments disclosed herein are given by way of example only and are not intended to limit the scope of the disclosure in any way.

What is claimed is:

1. A device for performing light therapy, the device comprising:
a catheter body configured for placement in a subject; and
a distal tip coupled to the catheter body, the distal tip including a light source and an expandable balloon, the light source being capable of emitting a sufficient amount of light through the balloon to perform light therapy, the balloon having a main body and at least one deployable tissue engaging element connected to the main body, the at least one deployable tissue engaging element extending outwardly from the main body when the balloon is expanded.

2. The device of claim 1 wherein the balloon is a spiky balloon formed by the main body and a plurality of deployable tissue engaging elements.

3. The device of claim 1 wherein the at least one deployable tissue engaging element is sufficiently rigid to penetrate fat.

4. The device of claim 1 wherein the light source comprises a light bar extending longitudinally through a chamber of the expandable balloon, the light bar comprising a plurality of selectively activatable light emitting devices.

5. The device of claim 1 wherein the at least one deployable tissue engaging element comprises an array of radially extendable members spaced from each other along the main body.

6. The device of claim 1 wherein the catheter body includes an inflation lumen through which fluid flows into a chamber of the balloon.

7. A method of treating a patient, the method comprising:
placing an expandable distal end of a catheter within the patient;
inflating the expandable distal end;
deploying at least one tissue engaging element of the distal end such that the at least one tissue engaging element extends outwardly from the distal end to engage tissue of the patient; and
emitting a sufficient amount of light from the expandable distal end to treat target cells in the patient.

8. The method of claim 7 wherein the expandable distal end and the at least one tissue engaging element form a spiky balloon.

9. The method of claim 7 wherein inflating the expandable distal end and deploying the at least one tissue engaging element comprises filling the expandable distal end with a fluid.

10. The method of claim 9 wherein the fluid is a light scattering fluid.

11. The method of claim 7 wherein the expandable distal end comprises a spiky balloon surrounding at least one light source capable of emitting the light.

12. A light delivery system comprising:
an insertion device having a distal end for placement in a subject and a longitudinally extending working lumen extending proximally from the distal end; and
a light delivery apparatus dimensioned so as to fit within the working lumen, the light delivery apparatus coupled to the working lumen such that the light delivery apparatus is substantially rotationally locked with respect to the insertion device while being slidably coupled with the working lumen in an axial direction.

13. The system of claim 12 wherein the light delivery apparatus has a distal tip, the distal tip including a light source and an expandable member, the light source is configured to produce a therapeutic amount of light, the expandable member has a collapsed configuration and an expanded configuration.

14. A device for performing light therapy, the device comprising:
a catheter body configured for placement in a patient;
an activatable light source configured to output a therapeutic amount of light energy;
an expandable member coupled to the catheter body and surrounding the activatable light source, the expandable member movable between a first configuration and a second configuration; and
a reflector coupled to the expandable member, the reflector facing the activatable light source such that, when the expandable body is in the second configuration, light energy delivered from the light source is reflected by the reflector.

15. The device of claim 14 wherein the reflector is movable with respect to the light source.

16. The device of claim 14 wherein the reflector is positioned with respect to the light source such that a substantial amount of the light energy delivered from the light source strikes the reflector.

17. The device of claim 14 wherein the reflector is moved away from the light source when the expandable member is moved from the first configuration to the second configuration.

18. The device of claim 14 wherein the activatable light source is a light bar comprising a plurality of light emitting devices.

19. The device of claim 14 wherein the expandable member comprises a transmissive portion positioned to transmit light reflected from the reflector.

20. The device of claim 19 wherein the light source is positioned between the transmissive portion and the reflector.

21. The device of claim 14 wherein the expandable member has a first portion to which the reflector is coupled, a second portion opposing the first portion, and a sidewall extending between the first portion and the second portion, and wherein, when the expandable member is in the second configuration, the light source is spaced from and between the first portion and the second portion.

22. The device of claim 14 wherein the expandable member has a generally planar shape when in the second configuration.

23. A method of treating a subject, the method comprising:
placing an expandable member in the subject, the expandable member coupled to a catheter body;
positioning a reflector coupled to the expandable member with respect to an activatable light source disposed within the expandable member;
delivering light from the light source towards the reflector; and
reflecting light from the activatable light source with the reflector such that the reflected light passes through a transparent section of the expandable member.

24. The method of claim 23, further comprising:
reflecting a therapeutically effective amount of the light from the light source with the reflector.

25. The method of claim 23 wherein a substantial portion of the light from the light source strikes the reflector.

26. The method of claim 23, further comprising:
inflating the expandable member with a flowable substance so as to move the reflector with respect to the activatable light source.

27. The method of claim 23 wherein placing the expandable member comprises orienting and positioning the expandable member with respect to targeted tissue such that the light reflected from the reflector illuminates the targeted tissue.

28. The method of claim 23 wherein a transmissive portion of the expandable member is at least proximate the targeted tissue while the reflector reflects light to the targeted tissue.

29. A light delivery apparatus for treating a subject, the apparatus comprising:
a flexible elongate device dimensioned for placement in a body of the subject, the elongate device having a first delivery configuration and a selected second configuration; and
an activatable light source positioned with respect to the elongate device such that, when the elongate device is in the second configuration, light from the activatable light source can illuminate a greater volume of tissue adjacent at least a portion of the elongate device than when the elongated device is in the first delivery configuration.

30. The light delivery apparatus of claim 29 wherein the activatable light source is capable of producing a sufficient amount of light to activate a therapeutically effective amount of a treatment agent in target tissue.

31. The light delivery apparatus of claim 29 wherein the elongate device in the second configuration has a generally serpentine shape.

32. The light delivery apparatus of claim 29 wherein the elongate device in the second configuration has a generally helical shape.

33. The light delivery apparatus of claim 29 wherein the elongate device in the second configuration has a generally spiral shape.

34. The light delivery apparatus of claim 29 wherein the elongate device has an array of articulatable fingers, at least one of the articulatable fingers movable between a collapsed configuration and an expanded configuration.

35. The light delivery apparatus of claim 34 wherein the array of articulatable fingers can penetrate fat.

36. The light delivery apparatus of claim 34 wherein the elongate device includes a tubular member and a plurality of deployable tissue engaging elements connected to the tubular member.

37. A method of performing light therapy comprising:
positioning a flexible elongate device in a subject;
moving the elongate device from a first configuration to a selected second configuration, wherein the elongate device in the first configuration is adapted to occupy a first volume, the elongate device in the second configuration is adapted to occupy a second volume that is greater than the first volume; and
delivering light energy from a light source through at least a portion of the elongate device in the second configuration.

38. The method of claim 37 further comprising:
administering a treatment agent to the subject; and
delivering a therapeutically effective amount of light from the light source to activate a therapeutically effective amount of the treatment agent.

39. A method of treating target tissue of a subject, the method comprising:
advancing an illumination device into the subject, the illumination device having at least one light source adapted to emit light capable of activating a treatment agent;
positioning the illumination device in a space in the subject, the space defined between a first layer of tissue and a second layer of tissue;
moving the illumination device from a first configuration to a selected second configuration; and
illuminating the first layer of tissue with light from the at least one light source.

40. The method of claim 39 wherein a substantial portion of the light from the at least one light source is directed towards the first layer of tissue, the first layer of tissue is the omentum and the second layer of tissue is the abdominal wall.

41. The method of claim 39 wherein the illumination device comprises a balloon movable between the first configuration and the second configuration.

42. The method of claim 39 wherein moving the illumination device from the first configuration to the selected second configuration comprises self-expanding the illumination device from the first configuration in which the illumination device occupies a first volume and the second configuration in which the illumination device occupies a second volume that is greater than the first volume.
